(19) United States
(12) Patent Application Publication
(10) Pub. No.: US 2009/0306640 A1
(43) Pub. Date: Dec. 10, 2009

(54) Vein Therapy Device and Method

(76) Inventors: Grant Michael Glaze, Sunnyvale, CA (US); Chun-Chih Cheng, Sunnyvale, CA (US); Jerome Jackson, Los Altos, CA (US); Joseph Michael Tartaglia, Morgan Hill, CA (US); Steven H. Trebotich, Newark, CA (US)

Correspondence Address: SHAY GLENN LLP, 2755 CAMPUS DRIVE, SUITE 210, SAN MATEO, CA, 94403, US

(21) Appl. No.: 12/480,536

(22) Filed: Jun. 8, 2009

Related U.S. Application Data
(60) Provisional application No. 61/059,518, filed on Jun. 6, 2008.

(51) Int. Cl. A61B 18/04

(52) U.S. Cl. 606/27

(57) ABSTRACT

A vapor delivery and insulation device is provided that may include any of a number of features. One feature of the device is that it can deliver vapor to the veins of a patient. The vapor can be generated within the device, in a handle of the device, or external to the device. Another feature of the device is that it can actively insulate the vapor to minimize heat transfer from a vapor delivery lumen of the device to an exterior surface of the device. The active insulation can be a vacuum or a flowing gas. Methods associated with use of the device are also covered.

### CROSS REFERENCE TO RELATED APPLICATIONS

This application claims the benefit under 35 U.S.C. 119 of U.S. Provisional Patent Application No. 61/059,518, filed Jun. 6, 2008, titled “Vein Therapy Device and Method.” This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The human venous system of the lower limb consists essentially of the superficial venous system and the deep venous system with perforating veins connecting the two systems. The superficial system includes the great saphenous, small saphenous and the lateral saphenous systems. The deep venous system includes the anterior and posterior tibial veins which unite to form the popliteal vein, which in turn becomes the femoral vein when joined by the short saphenous vein.

The venous systems contain numerous one-way valves for facilitating blood flow back to the heart. Venous valves are usually bicuspid valves, with each cusp forming a sack or reservoir for blood which, under pressure, forces the free surfaces of the cusps together to prevent retrograde flow of the blood and allow antegrade flow to the heart. When an incompetent valve is in the flow path of retrograde flow toward the foot, the valve is unable to close because the cusps do not form a proper seal and retrograde flow of blood cannot be stopped.

Incompetence in the venous system can result from vein dilation, which causes the veins to swell with additional blood. Separation of the cusps of the venous valve at the commissure may occur as a result. The leaflets are stretched by the dilation of the vein and concomitant increase in the vein diameter which the leaflets traverse. Stretching of the leaflets of the venous valve results in redundancy which allows the leaflets to fold on themselves and leave the valve open. This is called prolapse, which can allow reflux of blood in the vein. Eventually the venous valve fails, thereby increasing the strain and pressure on the lower venous sections and overlying tissues. Two venous diseases which often involve vein dilation are varicose veins and chronic venous insufficiency.

The varicose vein condition includes dilatation and tortuosity of the superficial veins of the lower limb, resulting in unsightly protrusions or discoloration, ‘heaviness’ in the lower limbs, itching, pain, and ulceration. Varicose veins often involve incompetence of one or more venous valves, which allow reflux of blood from the deep venous system to the superficial venous system or reflux within the superficial system.

Current varicose vein treatments include invasive open surgical procedures such as vein stripping and occasionally vein grafting, venous valvuloplasty and the implantation of various prosthetic devices. The removal of varicose veins from the body can be a tedious, time-consuming procedure and can be a painful and slow healing process. Complications including scarring and the loss of the vein for future potential cardiac and other by-pass procedures may also result. Along with the complications and risks of invasive open surgery, varicose veins may persist or recur, particularly when the valvular problem is not corrected. Due to the long, arduous, and tedious nature of the surgical procedure, treating multiple venous sections can exceed the physical stamina of the physician, and thus render complete treatment of the varicose vein conditions impractical.

Newer, less invasive therapies to treat varicose veins include intralumenal treatments to shrink and/or create an injury to the vein wall thereby facilitating the collapse of the inner lumen. These therapies include sclerotherapy, as well as catheter, energy-based treatments such as laser, Radio Frequency (RF), or resistive heat (heater coil) that effectively elevate the temperature of the vein wall to cause collagen contraction, an inflammatory response and endothelial damage. Sclerotherapy, or delivery of a sclerosant directly to the vein wall, is typically not used with the larger trunk veins due to treatment complications of large migrating sclerosant boluses. Laser energy delivery can result in extremely high tissue temperatures which can lead to pain, bruising and thrombophlebitis. RF therapy is typically associated with lengthy treatment times, and resistive heater coil treatments can be ineffective due to inconsistent vein wall contact (especially in larger vessels). The catheter based treatments such as laser, resistive heater coil and RF energy delivery also typically require external vein compression to improve energy coupling to the vein wall. This is time consuming and can again lead to inconsistent results. In addition, due to the size and/or stiffness of the catheter shaft and laser fibers, none of these therapies are currently being used to treat tortuous surface varicosities or larger spider veins. They are currently limited in their use to large trunk veins such as the great saphenous vein (GSV). Tortuous surface varicosities are currently treated with sclerotherapy and ambulatory phlembectomy, while larger spider veins are currently only treated with sclerotherapy.

What is needed is a therapy that addresses many of the shortcomings of current treatment modalities while providing safe, consistent and effective treatment. It should be safer and more consistent than laser or resistive heater coil therapy while not extending procedure time. It should be faster than and at least as effective as RF treatment. It also should eliminate the time-consuming process of compressing the vein walls. In addition, it is desirable to have a single therapy that has the potential to treat multiple types of veins including large trunk veins, tortuous surface varicosities and larger spider veins.

### SUMMARY OF THE INVENTION

One aspect of the invention is an elongate member configured to deliver vapor to treat a patient's vein, comprising, a vapor generator adapted to heat a liquid to generate vapor, a delivery lumen disposed in the elongate member, the delivery lumen being in fluid communication with the vapor generator, and an active insulation lumen disposed in the elongate member, the active insulation lumen being adapted to minimize heat transfer from the delivery lumen of the elongate member.

In some embodiments the active insulation lumen is coupled to a vacuum device, such as a vacuum pump or a wall vacuum. The vacuum device can be configured to actively create a vacuum in the active insulation lumen. In other embodiments, the active insulation lumen is coupled to a gas source.

The active insulation lumen can comprise an inflow portion adjacent to the delivery lumen and an outflow portion adjacent to an exterior surface of the elongate member. In another embodiment, the inflow portion can be adjacent to the exterior surface and in outflow portion can be adjacent to the delivery lumen. The inflow portion of the active insulation lumen can be coupled to a gas source or a vacuum source. In some embodiments, the delivery lumen and the active insulation lumen are not in fluidic communication.

In some embodiments, the vapor generator is disposed within the elongate member. In other embodiments, the vapor generator is disposed outside of the elongate member. In additional embodiments, the vapor generator is disposed in a handle on a proximal end of the elongate member.

In some embodiments, the delivery lumen and active insulation lumen are concentric. In other embodiments, the active insulation lumen is annular. In additional embodiments, an inflow portion of the active insulation lumen, an outflow portion of the active insulation lumen, and the delivery lumen are concentric. The active insulation lumen can also comprise a plurality of inflow and outflow lumens. The inflow and outflow lumens can be radially outward from the delivery lumen. In some embodiments, the inflow and outflow lumens can be approximately the same distance from the delivery lumen.

In yet another embodiment, the elongate member can further comprise a needle disposed on a distal end of the delivery lumen of the elongate member.

Another aspect of the invention provides a method of insulating vapor to be delivered to a patient, comprising, positioning an elongate member within a target tissue in the patient, delivering vapor through a delivery lumen of the elongate member to the target tissue, and actively forming a vacuum in an active insulation lumen of the elongate member to insulate the vapor.

In some embodiments, the delivering step further comprises generating the vapor in the delivery lumen. In other embodiments, the delivering step further comprises generating the vapor outside of the patient. In yet additional embodiments, the delivering step further comprises generating the vapor in a handle of the elongate member.

In some embodiments of the invention, the vacuum is formed with a vacuum device, such as with a vacuum pump or a wall vacuum.

In some embodiments of the method, the target tissue is a vein, an artery, or a gland duct.

In some embodiments of the method, the delivering step further comprises delivering vapor through a needle disposed on a distal end of the elongate member.

Another aspect of the invention provides a method of insulating vapor to be delivered to a vein in a patient's body, comprising, positioning an elongate member within the vein of the patient, delivering vapor through a delivery lumen of the elongate member to the vein, and actively insulating the vapor with an active insulation lumen of the elongate member.

In some embodiments, the actively insulating step comprises actively insulating the vapor with a vacuum in the active insulation lumen of the elongate member. The vacuum can be formed with a vacuum device, such as a vacuum pump or a wall vacuum, for example.

In other embodiments, the actively insulating step comprises actively insulating the vapor with a gas in the active insulation lumen of the elongate member. In some embodiments, the gas flows in a distal direction through the active insulation lumen adjacent to the delivery lumen and returns in a proximal direction through the active insulation lumen adjacent to an external surface of the elongate member. In other embodiments, the gas flows in a distal direction through the active insulation lumen adjacent to an external surface of the elongate member and returns in a proximal direction through the active insulation lumen adjacent to the delivery lumen.

In another embodiment, the actively insulating step comprises actively insulating the vapor with a fluid in the active insulation lumen of the elongate member.

In some embodiments, the vapor is generated in the delivery lumen. In other embodiments, the vapor is generated outside of the patient. In additional embodiments, the vapor is generated in a handle of the elongate member.

In another aspect of the method, the delivering step further comprises delivering vapor through a needle disposed on a distal end of the elongate member.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings wherein:

FIG. 1 illustrates an embodiment in which a heater coil is disposed at the distal end of an elongate member to generate vapor.

FIGS. 2A-2D illustrate cooled shaft embodiments of the invention.

FIGS. 3A-3E illustrate cooled shaft embodiments of the invention.

FIG. 4 illustrates an exemplary insulation sheath.

FIG. 5A and 5B illustrate an exemplary insulation sheath.

FIGS. 6A and 6B illustrate the distal region of an exemplary elongate member.

FIGS. 7A and 7B illustrate the distal region of an exemplary elongate member.

FIG. 8 shows an exemplary deflector for deflecting vapor.

FIGS. 9A-9C show a rotatable deflector for deflecting vapor.

FIGS. 10A-10D show exemplary designs for the distal region of an elongate member for delivering vapor.

FIG. 11 shows an exemplary method to confine the vapor to a particular volume.

FIG. 12 shows an inflatable balloon for confining the vapor within the vein.

FIG. 13 shows a distal and proximal balloons for confining the vapor within the vein.

FIG. 14 shows a slidable device with which to advance and manipulate a hot catheter shaft.

FIGS. 15A-15D illustrate an embodiment including a needle for delivering vapor to tissue.

### DETAILED DESCRIPTION OF THE INVENTION

The invention relates generally to systems and their methods of use to treat venous insufficiency. More particularly, the invention relates to vapor treatment of a vein to reduce its inner diameter to minimize and/or eliminate blood flow through the vein. The invention is generally used to divert the flow of blood from an insufficient vein to a vein that is sufficient.

The vapor treatments described herein can be used to treat any vein, such as trunk vessels (e.g., a great or small saphenous vein), sub-truncal veins (e.g., accessory vessels) or spider veins. The veins treated need not be varicose, however this it typically the case. The invention is not, however, limited to the treatment of the veins and the anatomical locations of the veins that are described herein. For example, the invention can be used to treat veins outside the leg region, such as abdominal varicosities, hemorrhoids, varicoceles, etc.

The treatments described herein generally include generating and delivering relatively high temperature (e.g., without limitation, greater than 37° C.) vapor through a delivery device to the lumen of a vein to reduce the inner diameter of the vein. A significant benefit of vapor delivery to reduce the lumen of the vessel is that it flows to the internal surfaces of the vein due to the increased pressure of the vapor and does not require external compression of the vein to enhance energy transfer of the device to the vein wall. Another significant benefit of the vapor delivery is the large amount of energy released in the transition of the vapor into the fluid phase. A further significant benefit of the vapor is that it is self-limiting in that it ceases to conduct heat to the vessel wall once temperature equilibrium has been reached between the vapor and the vessel wall. This is unlike other treatments which will continue to deliver energy to the tissue to the point of extensive thermal injury.

The vapor (such as steam) can be generated in a variety of locations in the system. For example, the vapor can be generated in a remote boiler or control console separate from the delivery device, within a handle or handpiece, or within the portion of the elongate member (such as a catheter) that is inserted into the vein. The vapor can be generated in any portion of the elongate member that is either inside or outside of the patient, for example.

In embodiments in which the vapor is generated in a remote boiler or console, the entire vapor path up to the disposable catheter hub is preferably sterilizable, either via an inserted sterile liner (similar to disposable baby bottle inserts along with associated flexible tubing and access ports) or a sterilizable pathway.

There are multiple ways in which vapor can be generated in the handle. For example, a resistive heater coil could be used that heats fluid as it flows around and past it to the elongate member. An inductive heater coil could also be used which creates an electromagnetic field within the coil to heat a tube or wire within which then heats flowing fluid and converts it into steam. A microwave source or laser (light energy) source could also be incorporated into the handle or handpiece which directly heats the fluid flowing through it and converts it into steam.

The vapor can also be generated within the elongate member (e.g., catheter). In some of these embodiments the steam is generated at the elongate member tip, or at a distal region of the elongate member. In other cases the steam can be generated anywhere within the shaft of the elongate member, not merely at the tip. The vapor can be generated in the catheter by flowing fluid through or around a hot element, such as a laser fiber or a target made hot by shining light energy on it. The hot element could also be made hot by flowing current through it, as in a resistive coil configuration. In addition, the amount of heat imparted to the flowing fluid could be increased by increasing the surface area of the hot element, for example by using multiple “spheres” or “fins”. Also, fluid could flow through a hot tube until enough heat has been imparted with which to turn the flowing fluid into vapor.

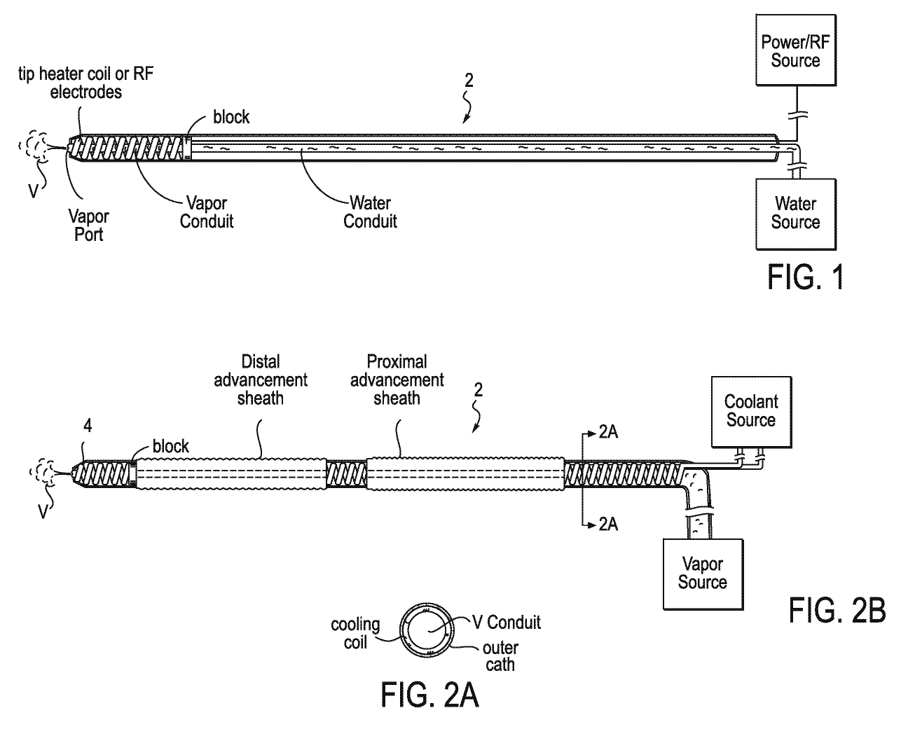

FIG. 1 illustrates an exemplary embodiment in which a heater coil is disposed at the distal end of the elongate member 2, such that as the fluid (shown as water) flows within or around the coil, vapor (“V”) is generated and delivered from the vapor port.

Alternatively, a conductive fluid can be directed past tip-based RF electrodes, thereby instantly heating and vaporizing the fluid. The current directed to the electrodes would conduct through the nearby flowing fluid, thereby causing vaporization of that fluid. An inductive heater coil could also be incorporated into the catheter body or tip which creates an electromagnetic field within the coil to heat a tube or wire within which then heats a fluid flowing through it and converts it into steam. A microwave source or a laser (light energy source) could also be incorporated into the catheter body or tip which directly heats the fluid flowing through it and converts it into steam.

Delivery of the fluid to the catheter tip prior to heating can be passively controlled via capillary action by utilizing various porous media, such as a wick, sponge, sintered polymer or metal structure, or via other wicking methods. The fluid can also be actively delivered by pressurizing the fluid, thereby causing forward flow through a delivery tube. The pressure source can be passive, as in an elevated bag, or it can be active, as in a positive displacement pump, peristaltic pump or other pumping method.

The power sources for generating the vapor can be any electromagnetic waves such as RF, microwave, infrared, ultra-violet (UV), laser, etc. Direct Current (DC), such as a battery, or Alternating Current (AC) can also be used. The vapor can be generated using any form of heat transfer, including conduction (e.g., resistive heaters, inductive heaters, etc.), convection (e.g., heat exchangers, etc.), and radiation (e.g., UV, etc.), or the fluid can be directly heated using microwave or ultrasound energy delivery.

The shaft of an elongate delivery member that delivers hot vapor to its distal tip will likely become hot along its length. It may become too warm for the care-giver to touch, or also cause inadvertent heating of an unintended target on the patient, e.g., a vein segment not indicated for treatment, or the patient's skin. This may result in burning of the skin in situations where the catheter shaft is directly exposed to the skin's surface, or where the catheter shaft is disposed in a vein located close to the surface of the skin.

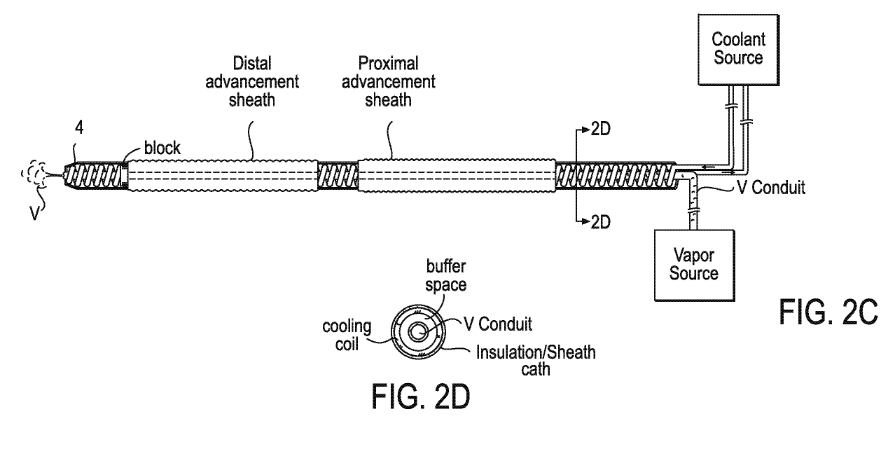

Thus, in some embodiments the elongate member has an insulation system to prevent the exterior surface of the elongate member from becoming too hot, while allowing the vapor being delivered to retain its heat during delivery. FIGS. 2, 2A, 2C and 2D illustrate exemplary cooled shaft embodiments in which a tube 4 coiled either along the elongate member 2 outer diameter, or extruded within the elongate member wall itself, provides a conduit for cooling fluid or gas to pass through or for a vacuum to be created within. The cooling effect could be further augmented with the addition of a ‘buffer’ space, as specifically shown in FIG. 2D.

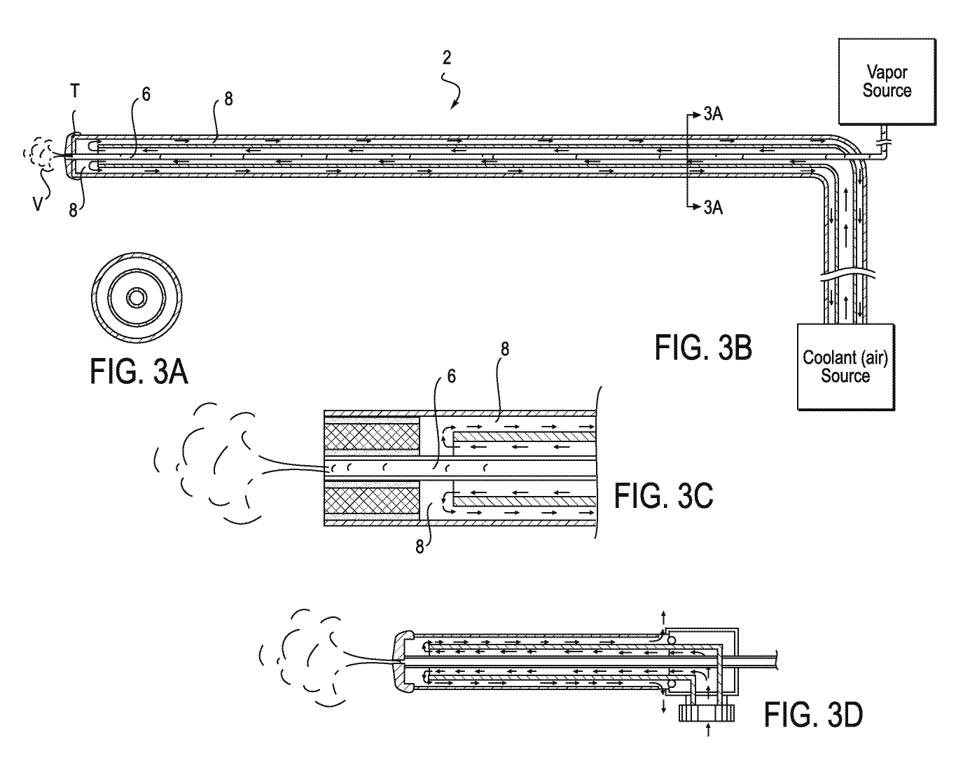

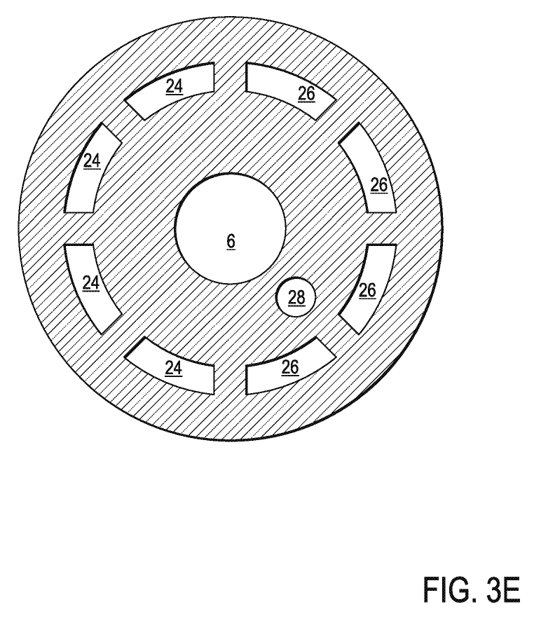

In the embodiment shown in FIGS. 3A-3D, the elongate member 2 includes a vapor delivery lumen 6 and an active insulation lumen 8. A gas, such as air, flows through the insulation lumen and reduces the amount of heat transferred from the vapor through the elongate member wall to the exterior shaft, thereby reducing the temperature of the exterior surface of the elongate member. In some embodiments, the gas can comprise a gas with better insulative properties than air (e.g., the gas can have a lower thermal conductivity, a lower heat capacity, or a lower mass than air). Suitable gases may include nitrogen, carbon dioxide, freon, argon, krypton, sulphur hexafluoride (SF6), halogenated hydrocarbon refrigerants (CCl₄, CCl₂F₂, CCl₃F, or C₂Cl₂F₄), or other appropriate gases.

In another embodiment, the space defined by the insulating lumen can be evacuated (i.e., create a vacuum in the active insulation lumen) to minimize heat transfer from the delivery lumen to the exterior surface of the elongate member, thereby reducing the temperature of the exterior surface of the elongate member. A vacuum can be actively created in the active insulation lumen with any vacuum device, such as a vacuum pump or a wall vacuum, for example.

In FIGS. 3A-3D, the active insulation is shown flowing in a distal direction in an inflow portion of the active insulation lumen adjacent the vapor delivery lumen, and then returning back towards the proximal end of the elongate member in an outflow portion of the active insulation lumen. In an alternative embodiment, the active insulation gas can flow in a distal direction through the lumen next to the external surface of the elongate member, and then return back towards the proximal end of the elongate member through the lumen adjacent the vapor delivery lumen for enhanced thermal performance (i.e. the cooler air flows through the external loop closest to the skin and keeps the skin cooler, and the warmed air flows in a counter flow direction back out the proximal end of the elongate member which reduces pre-cooling of the vapor). The vapor delivery lumen and the active insulation lumen are not in fluidic communication. As shown in FIG. 3D, the gas that is flowing proximally can be expelled from the delivery system near a proximal portion of the elongate member. Alternatively, the inlet can be closed and the space evacuated to reduce the amount of heat conducted to the external wall. In addition, the elongate member can also have an outer insulation layer (which can be a sheath) as shown in FIGS. 3A.

As shown in FIG. 3A, the delivery lumen and active insulation lumens can be concentric. In some embodiments, as described above, the active insulation lumen can be annular. In another embodiment, as shown in FIG. 3E, the active insulation lumen can comprise a plurality of active insulation lumens 24 and 26. In the embodiment of FIG. 3E, inflow lumens 24 and outflow lumens 26 are positioned radially outward from the delivery lumen 6. The inflow and outflow lumens can be approximately the same distance from the delivery lumen, for example. In FIG. 3E, the active insulation path can flow distally towards a distal end of the elongate member along inflow lumens 24, and then flow proximally through the elongate member along outflow lumens 26. The elongate member may also include an electrical wire lumen 28 to power any electrical devices, such as electrodes, in the elongate member, for example. The elongate member shown in FIG. 3E could be made from a single multi-lumen extrusion, for example. In even other embodiments, the active insulation lumens can be spirally traversing lumens (not shown).

In another embodiment, the shaft would be allowed to get hot, and an insulating sheath would provide surface skin protection to the care-giver and patient. The portion of the elongate member surrounded by the insulating sheath could be the portion that remains external to the patient (which would likely be held by the caregiver and which could also burn the patient if that portion of the catheter came into contact with patient skin). A portion of the elongate member surrounded by the insulating sheath could also be partially inside the vein of the patient. The insulating sheath would be made of a material suitable for mitigating temperatures too hot to touch and/or are capable of burning the skin. For example, the material could be a polymer such as silicone or a material coated in ceramic.

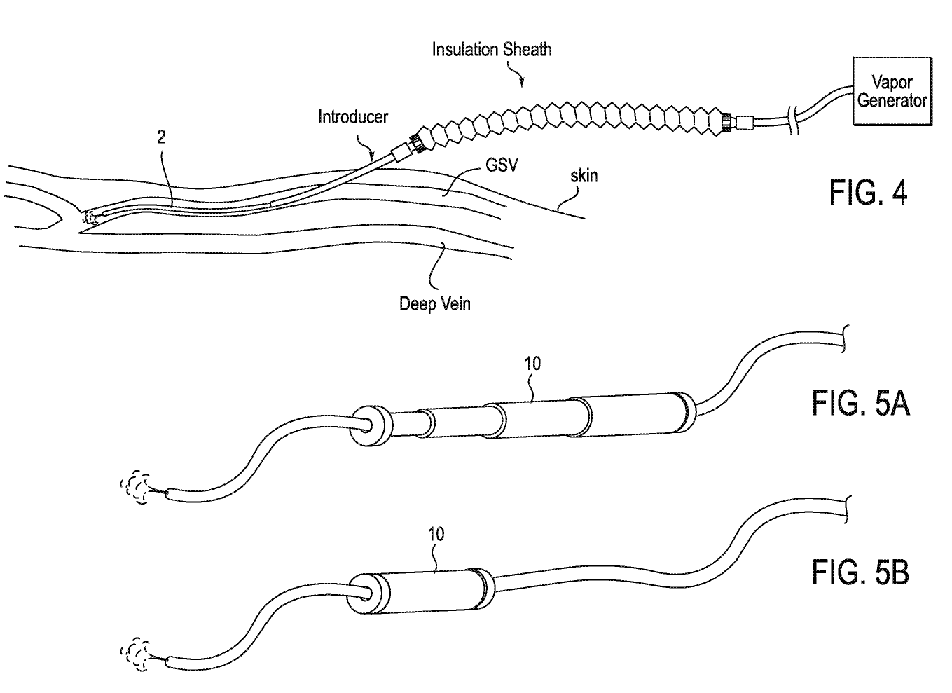

The sheath can be adjustable in length, as the elongate member will likely be retracted proximally within the vein during the treatment. FIG. 4 illustrates one embodiment of an “accordion” style insulation sheath. The insulation sheath can be used with a standard introducer sheath, and the sheath collapses as the catheter 2 is inserted, up to a maximum amount that coincides with an entire elongate member insertion (e.g., at the sapheno-femoral junction).

In an alternative embodiment shown in FIGS. 5A and 5B, a telescoping style insulating sheath 10 could be employed. This sheath would attach to either a standard or other introducer sheath, and it would provide insulating characteristics similar to the accordion style sheath. It would telescope inward when the catheter has been fully inserted into the vein to be treated, overlapping on each successive section, and then extend upon retraction during treatment.

The catheter tubing material generally needs to be able to deliver high temperature vapor to a target location within the patient and provide enough flexibility for delivering the catheter along a tortuous route without collapsing. Traditional catheters are typically made from thermoplastic materials that provide the right physical characteristics, e.g.: flexibility and kink resistance, to facilitate advancement through tortuous vasculature. However, thermoplastic materials are generally not suitable for elevated temperature operation since their physical characteristics typically change substantially with elevated temperature. Thermoset materials can provide the right elevated temperature resistance, but they are typically not suitable for catheter applications (e.g. silicone and other rubber compounds are thermoset materials but they do not have the structural properties, e.g., column strength and flexibility, that allow them to be used in such a configuration). Other thermoset materials that have the required structural characteristics for advancement through vessels typically cannot be advanced around tight bends, and are hence undesirable in this type of catheter shaft.

In some embodiments the catheter comprises a thermoset composite material which has the benefit of its properties not being significantly altered at high temperature and also has the ability to be delivered around tight bends and tortuous paths. The tubing used in the catheter's construction is a composite thermoset material which is reinforced with a braid material. The composite thermoset material can be, for example, polyimide or other similar material. The braid can be a metal, such as stainless steel or other similar material, or a plastic, such as polyester, nylon, or other similar material. The braid allows the tubing to be bent around tight radii without collapsing.

The reinforcement component could also take on other forms, such as a coil rather than a braid. A coil design has the added benefit of reducing the overall profile, as the individual strengthening filaments don't need to cross over each other. The compressive strength and incompressibility of a coil are generally better than a braid, enhancing the crushability of the catheter. A catheter with a coil would be flexible (perhaps more flexible than a catheter with a braid), less crushable, more pushable, and would provide more kink resistance.

For delivering the vapor from the elongate member, the elongate member includes at least one vapor delivery port along its length. The at least one vapor delivery port is typically disposed at or near the distal end of the elongate member. The size, shape, and location of the vapor port(s) will impact the angle at which the vapor is delivered from the elongate member.

Inadvertent heating of vein tissue distal to the target tissue (the vein tissue intended to be treated) may result from a catheter whose distal end is open. This could lead to dispensing the vapor too far distal to the target tissue. A tip that controls, deflects, diverts, or reduces the amount of vapor exiting the vapor delivery port could mitigate this potential issue.

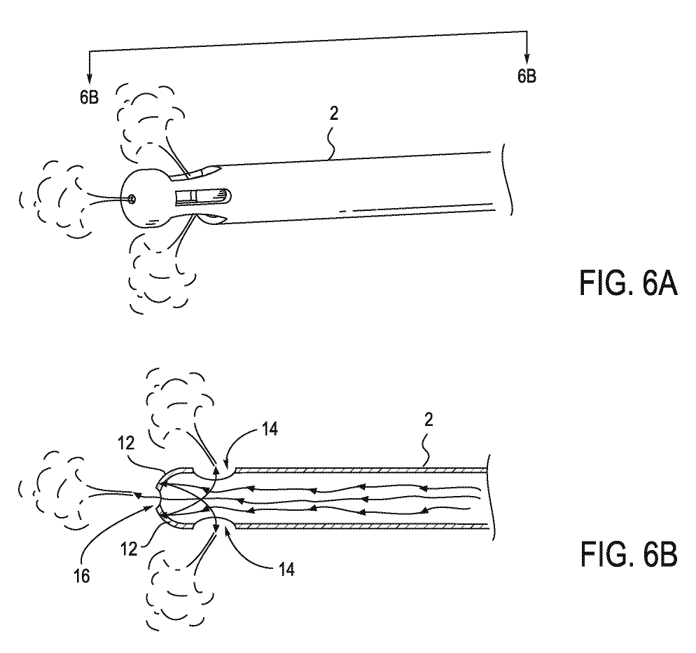

In some embodiments, the elongate member includes a deflector 12 which causes the vapor that comes into contact with the deflector to be deflected from the sides of the elongate member as opposed to delivering the vapor in the distal direction. In the embodiment shown in FIGS. 6A and 6B the side ports 14 are larger than the distal port 16. In this embodiment most of the vapor is deflected out of the sides of the elongate member, while only a small amount of vapor is delivered through the distal port. The distal end could also be closed off such that the elongate member does not have a distal port and all the vapor is deflected out of the side ports.

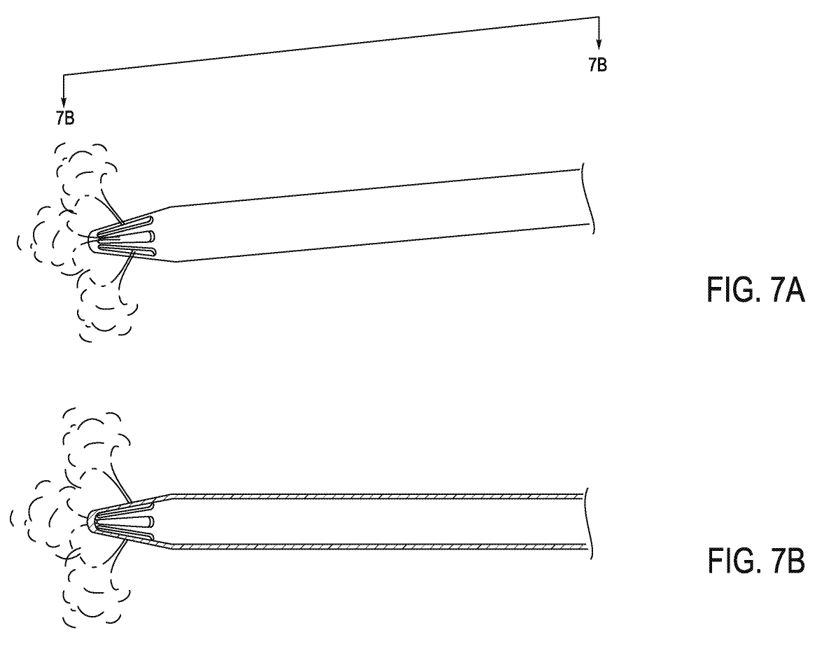

An alternative “bullet” design shown in FIGS. 7A and 7B has a closed-tip configuration. The vapor ports can be adapted to influence the direction in which the vapor flows from the elongate member. For example, the ports can be tear-drop in shape (e.g., the large portion of the tear-drop shape disposed at the proximal end of the vapor port) so that more vapor escapes sideways instead of forward. The slots can have a cone-like configuration as well.

The vapor delivery port(s) can also be adjustable in diameter, shape, or orientation. The number of vapor delivery port(s) can also be varied to uniformly deliver vapor to the vessel wall (e.g. 2, 3, 4, etc., or a multitude of ports such as a diffuser or shower head). The elongate member can also have an adjustable element within the vapor delivery lumen which alters the deflection of the vapor out of the elongate member. An adjustment element in the handle of the delivery system can allow a user to actuate an adjustable element disposed in the distal region of the elongate member to either change the shape of the vapor port or to change the orientation of a deflector located within the elongate member. This adjustability allows the user to deliver the vapor differently based on the location within the vein or the type of vein being treated. For example, the distal vapor port could be in a closed configuration when the distal end of the elongate member is placed near the sapheno-femoral junction to prevent undesired deep vein treatment. The distal vapor port could then be changed to an open configuration once the distal end is clear of the deep veins. In addition, the side ports could be closed off and the distal port could be changed to a fully open configuration to minimize the amount of vapor delivered from the side of the elongate member. This could be desirable when near, for example, a competent perforator.

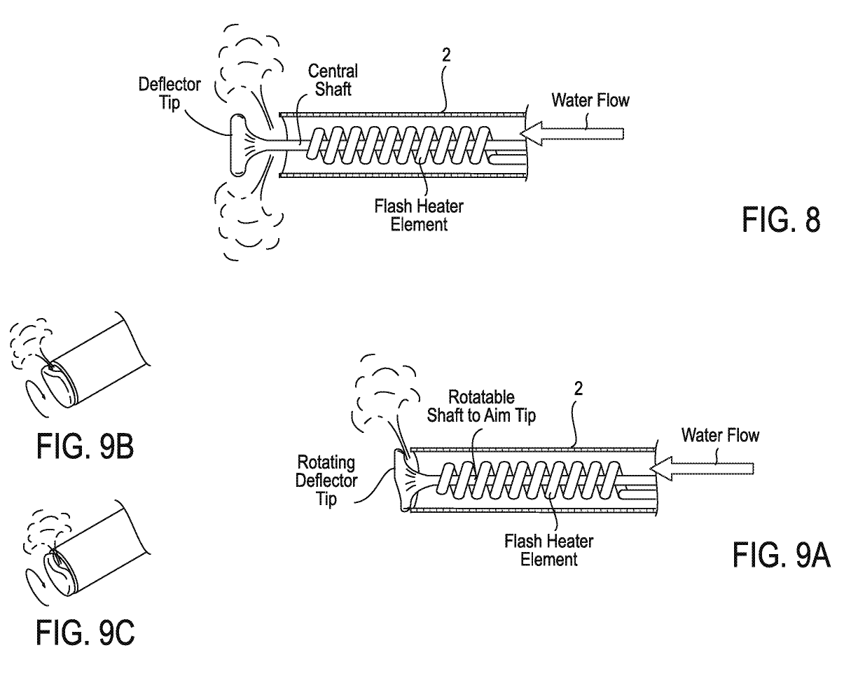

FIG. 8 illustrates an embodiment in which liquid flows through the catheter or other elongate member and is vaporized at the distal exit port by a flash heater element. A deflector comprising a deflector tip supported by a central shaft in the elongate member deflects the exiting vapor radially. FIGS. 9A-9C show a similar embodiment in which the deflector is asymmetric and can be rotated, such as by actuating an actuator at a proximal end of the elongate catheter or in a handpiece (not shown). Rotation would facilitate directional flow of vapor to a specific radial target, e.g. a perforator vein.

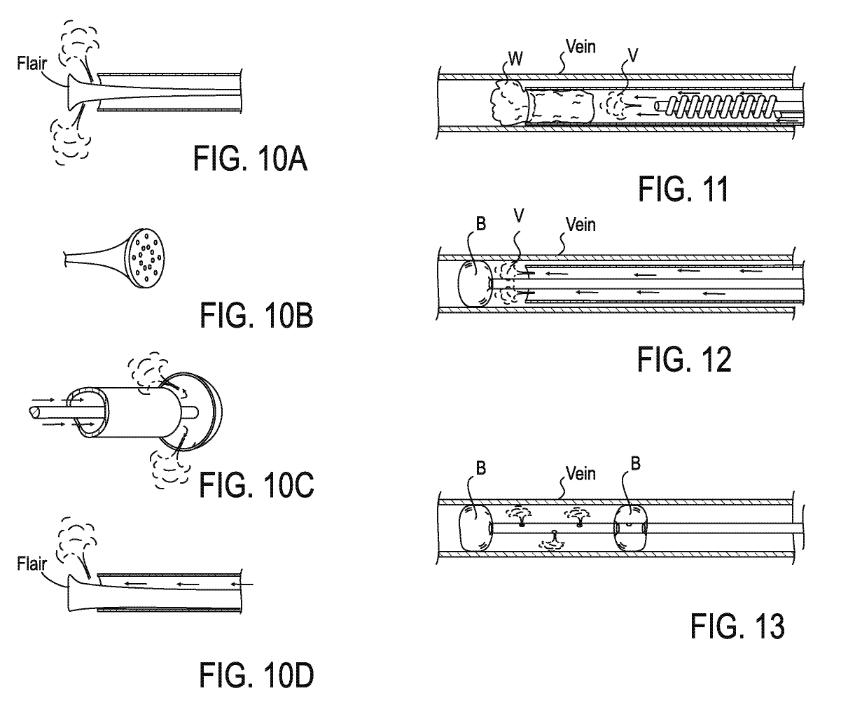

FIGS. 10A-10D show alternative designs for vapor delivery at the distal region of the elongate member: a flared-tube design (FIG. 10A), either circumferentially consistent in shape or preferentially configured to one side; a shower-head design (FIG. 10B), either omni-directional or having an adjustable spray direction (alternatively, the plurality of shower head ports can be disposed on the side of the elongate member rather than facing in a distal direction); an umbrella design (FIG. 10C) that acts as a hard stop, thereby preventing any steam flow in the direction blocked by the umbrella footprint; and a curved tube array design (FIG. 10D) providing a finite stream of steam delivered in a specific direction governed by the end shapes of the tube.

In some embodiments, it may be desirable to confine the vapor to a particular volume within the vein. In FIG. 11, a plug of fluid (such as water “W”) is delivered prior to delivery of the vapor. The fluid plug confines the delivered vapor to the volume between the plug and the distal portion of the delivery tool. The plug of fluid can also be vaporized upon the application of energy to the fluid plug.

In FIG. 12, an inflatable balloon “B” is placed distal to the vapor outlet to confine the vapor to a location proximal to the balloon. The balloon can be inflated as either a separate step in the procedure or it can be automatically inflated using the steam delivered to treat the vein tissue.

FIG. 13 shows an embodiment with a two-lobed balloon providing proximal as well as distal confinement of the vapor. Two separate balloons may also be used.

In some embodiments, the system can have a detachable end effector that is adapted to be reversibly coupled to the distal end of the elongate member. Thus, a plurality of end effectors with a variety of exit ports can be used with a single elongate member, depending on the type of vein tissue is being treated, the desired delivery direction of the vapor, etc.

The distal region of the elongate member (also described herein as an end effector) can have 1, 2, 3, 4, or more vapor delivery ports. The elongate member can include almost any number of ports. The ports can be disposed in almost any orientation around the elongate member, but in one embodiment the ports are circumferentially equidistant. For example, in one embodiment there are two vapor ports that are 180 degrees apart from each other around the circumference of the distal region of the elongate member. A different elongate member can have four vapor ports substantially 90 degrees apart from one another around the circumference of the elongate member.

In some embodiments the vapor delivery ports are substantially circular in shape, which delivers the vapor from the elongate member at substantially 90 degrees to the longitudinal axis of the elongate member. This allows for vapor delivery to the tissue closest to the vapor port, which allows for the most amount of heat transfer from the vapor to the vein tissue.

The vapor ports can be matched to the inner catheter diameter, such that either less or more surface area is created for vapor to exit. For instance, the summation of port surface area can be less than the vapor delivery lumen cross-sectional area, thereby causing a “nozzle” effect at the distal end. Alternatively, the cumulative port surface area can be matched to or greater than the inner vapor delivery lumen cross-sectional area, thereby facilitating unrestricted vapor outflow.

A method of insulating vapor to be delivered to a patient will now be described. The method can include positioning an elongate member within a target tissue in the patient. The elongate member can be any elongate member described herein. The target tissue can be a vein, an artery, a gland duct, or any other similar structures such as hollow or tubular organs.

The method can further include delivering vapor through a delivery lumen of the elongate member to the target tissue. In some embodiments, the vapor is generated in the delivery lumen. The vapor can be generated in the delivery lumen with electrodes or heated coils positioned near a distal end of the delivery lumen, for example. In other embodiments, the vapor can be generated outside of the patient, such as in a remote boiler. In yet other embodiments, the vapor can be generated in a handle of the elongate member.

The method can further include actively forming a vacuum in an active insulation lumen of the elongate member to insulate the vapor. The vacuum can be actively formed with a vacuum device, such as with a vacuum pump, wall vacuum, or other similar device.

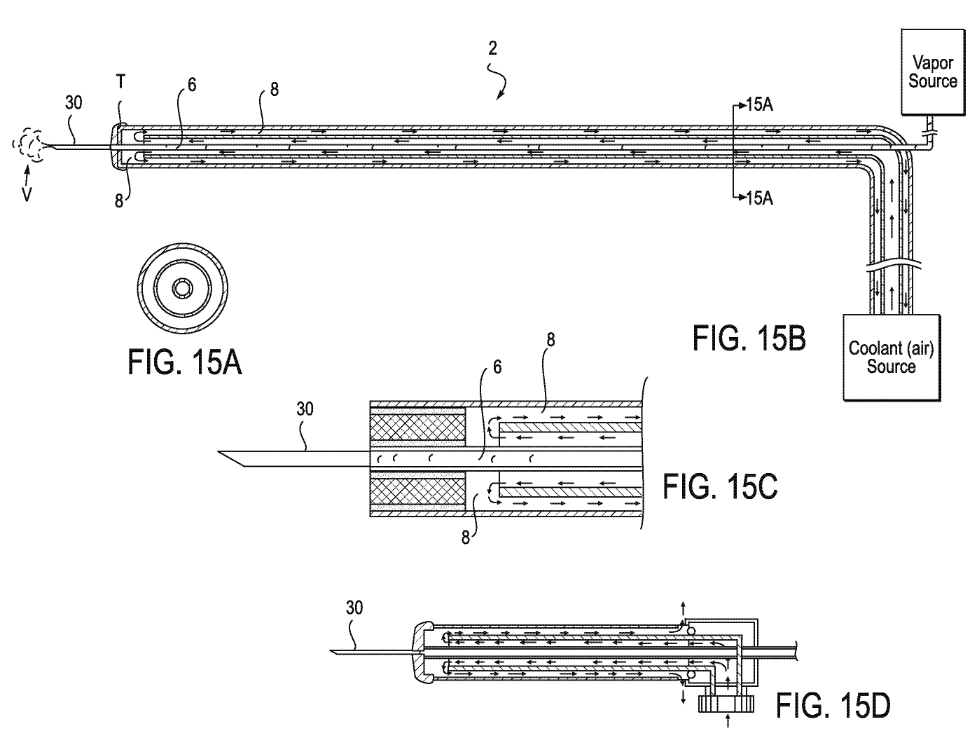

In yet another embodiment of the method, the vapor can be delivered through a needle 30 disposed on a distal end of the elongate member, as shown in FIGS. 15A-15D. The vapor can be delivered through the delivery lumen and through the needle 30 to tissue.

To treat the entire length of a vein (by transferring heat from the vapor to the vein tissue), it may be necessary to move (either proximally or distally) the elongate shaft during the procedure to treat all of the vein tissue. The elongate member may be moved substantially proximally continuously as vapor is delivered from the vapor port, or it may be move incrementally, such that a period time passes between movements of the elongate member. Exemplary pullback speeds that can be used include about 2 cm/sec, about 1 cm/sec, about 0.5 cm/sec, and about 0.33 cm/sec, however many combinations of pullback speeds and vapor delivery volumes can be used. Vapor pressures that can be used include pressures of about 20 psi to about 70 psi, however other pressures can be used depending on the internal diameter of the vapor delivery catheter and the vapor delivery ports. Energy released from the vapor to liquid phase transition reduces the inner diameter of the vein during the treatment due to, e.g., collagen shrinkage, endothelial denudation, and the inflammatory response.

The treatment parameters (such as vapor pressure and pullback speed, etc.) can be adjusted based on the diameter of the vein being treated. A larger vein may require additional pressure and/or a slower pullback speed such that the vein tissue is treated sufficiently. Similarly, a smaller diameter may necessitate less pressure and/or a faster pullback speed to avoid damaging tissue surrounding the vein, such as nerves. Moreover, partial duty cycles (e.g. 50%) can be used with which to further reduce energy delivery to the vein wall.

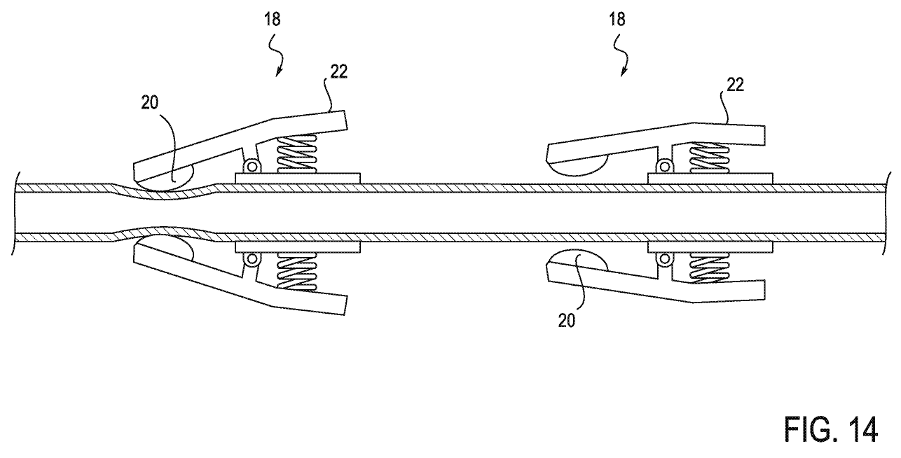

FIG. 14 shows a feature that will enable users to control movement of the elongate member within the vein. One or more collet-type mechanisms 18 are disposed on the shaft of the elongate member, each having a distal gripper ring 20 that provides a compression force to the shaft in one configuration and does not provide the force in a second configuration. When the ring is not in contact with the shaft (e.g., when a user actuates surface 22), the user can move the gripper proximally or distally along the length of the shaft. This device also allows the user to indirectly contact a catheter shaft, should the shaft be too hot to touch.

The gripper shown in FIG. 14 can also be adapted so that a user can actuate the gripper to move the catheter shaft relative to the gripper, such as during the deliberate movement of the elongate member during the vapor delivery. For example, the gripper can comprise a ratchet mechanism. The actuation element can be adapted to move the elongate member a specific distance each time the gripper is actuated, e.g. 1 cm, allowing for precise and controlled movement of the elongate member by the user.

The management of the amount of energy applied by the vapor to the tissue (dose management) depends on where the vapor is generated. If the vapor is generated at a remote boiler, the energy delivered can be described in terms of temperature and pressure via sensors located in the remote boiler. The fluid flow rate can also be controlled with the boiler. If the vapor is generated at the handpiece, temperature and flow rate can be obtained with sensors and can detect information about the energy being delivered. If vapor is generated at the catheter tip, the fluid flow rate and power delivered to the heating device could be used to control the energy delivered. Alternatively, the power delivered to the heating device can be controlled to vaporize all the fluid supplied to the tip such that and the energy delivery calculated accordingly. The temperature can be pre-determined and calibrated into the system. A temperature sensor at the tip can determine the temperature of the vapor and the flow rate can be adjusted accordingly.

The length of the vapor delivery lumen can have an impact on the temperature of the vapor as it exits the vapor ports. The longer the vapor delivery lumen, the lower the temperature of the vapor will be as it exits the device. The vapor loses energy as it passes through the delivery lumen by transferring energy to the delivery lumen wall. One way to maintain a desired vapor temperature is to increase the inner diameter of the vapor delivery lumen. Increasing the diameter can increase the amount of steam that arrives at the distal end of the delivery lumen in the same amount of time, which provides steam with a higher temperature. For example, a catheter with vapor delivery lumen that has an inner diameter of about 0.033 in and a length of about 45 cm will deliver adequate quantities of vapor at an appropriate temperature. However, if the length is increased to about 75 cm, an inside diameter of about 0.044 in may be needed to deliver a similar quantity of vapor at a similar temperature. The vapor delivery lumen inside diameter and length as well as the vapor pressure can be varied to provide many combinations that provide an adequate quantity of vapor at an appropriate temperature.

Another way to maintain a desired vapor temperature is to heat the wall of the vapor delivery catheter or lumen along its length such that the temperature of the catheter wall or vapor delivery lumen approaches that of the vapor. In this case, vapor temperature and quality changes would be minimized as it traverses the length of the catheter since the losses it experiences due to the length it travels would be minimized. Such heating can be accomplished by inclusion of a resistively heated coil along the length of the catheter or vapor delivery lumen. Alternatively, an additional lumen can be provided along and directly adjacent to the vapor delivery lumen which contains a liquid or gas at an elevated temperature to minimize the heat loss from the vapor delivery lumen. The additional lumen can also contain air or a vacuum and provide an additional barrier to heat loss by the vapor delivery lumen.

One aspect of the invention can use vapor to specifically treat surface varicosities which cannot be treated with currently available laser, RF or heater coil therapies. Typically when treating small superficial veins or surface varicosities, a catheter-based system is not used because it is difficult to advance a catheter in small diameter or tortuous vessels. Furthermore, energy-based treatment is not used because it is difficult to prevent skin burns in these superficial veins as the protective layer of tumescent fluid (either circumferentially consistent in shape or preferentially configured to one side with which to provide enough of a heat sink between the vessel and the skin surface) can not be administered.

Small superficial veins and/or surface varicosities can be accessed similarly to standard sclerotherapy techniques. However, rather than placing a sclerotherapy needle inside the vein to deliver a sclerosant fluid, a needle (e.g., needle 30 in FIGS. 15A-15D) adapted to deliver vapor would be placed in the vein. The needle can be disposed on a distal end of the Attorney Docket No.: 10653-700.200 delivery lumen of the elongate member described herein, for example. Once the vapor is generated and delivered out of the tip of the needle, it can easily travel through the vein and successfully traverse the tortuosities; catheter or needle access along the entire desired treatment length need not be achieved. Thus, a single, or a reduced number of needle sticks are required to treat an extensive network of small or tortuous superficial veins. The needle can be a standard hypodermic needle or it can include other vapor ports such as those described above. The outer shaft of the needle can be insulated, either passively by incorporating a low heat conducting material onto its outer surface or by including an active cooling insulating sleeve as described for the catheter delivery method described above.

To provide a heat sink with which skin burns can be prevented, a standard cold pack can simply be placed on the skin's surface, directly over the veins being treated. A cold pack may, however, absorb too much of the vapor energy delivered to the vein due to its close proximity to the vessel being treated. Therefore, a thermally-absorbent pack (not necessarily cold) can be placed on the skin that would absorb enough energy to prevent a skin burn yet not absorb too much energy such that the therapy to the vein is ineffective. This would enable vapor treatment of a small or tortuous superficial veins without burning the skin. This thermally-absorbent pack could, for instance, simply be at room or body temperature.

One alternative method of treatment would be to heat the fluid in a pre-filled vein such that the fluid turns into vapor within the vein. The fluid could be actively deposited by delivering saline or other biocompatible fluid, or could be residual blood in the lumen. A heating method, as previously discussed, incorporated into the distal end or tip of the catheter could provide sufficient energy to vaporize this fluid and provide the therapeutic effect. Obstructing members (such as balloons) could be disposed distally and proximally of the treatment area to prevent the loss of the resident fluid, and/or prevent the flow of vapor outside of the intended treatment area. This ‘resident’ fluid could be placed by injection through an elongate member and immediately prior to energy delivery.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. An elongate member configured to deliver vapor to treat a patient's vein, comprising;
a vapor generator adapted to heat a liquid to generate vapor;
a delivery lumen disposed in the elongate member, the delivery lumen being in fluid communication with the vapor generator; and
an active insulation lumen disposed in the elongate member, the active insulation lumen being adapted to minimize heat transfer from the delivery lumen of the elongate member.

2. The elongate member of claim 1 wherein the active insulation lumen is coupled to a vacuum device.

3. The elongate member of claim 2 wherein the vacuum device is a vacuum pump.

4. The elongate member of claim 2 wherein the vacuum device is a wall vacuum.

5. The elongate member of claim 2 wherein the vacuum device is configured to actively create a vacuum in the active insulation lumen.

6. The elongate member of claim 1 wherein the active insulation lumen is coupled to a gas source.

7. The elongate member of claim 1 wherein the active insulation lumen comprises an inflow portion adjacent to the delivery lumen and an outflow portion adjacent to an exterior surface of the elongate member.

8. The elongate member of claim 7 wherein the inflow portion of the active insulation lumen is coupled to a gas source.

9. The elongate member of claim 1 wherein the delivery lumen and the active insulation lumen are not in fluidic communication.

10. The elongate member of claim 1 wherein the vapor generator is disposed within the elongate member.

11. The elongate member of claim 1 wherein the vapor generator is disposed outside of the elongate member.

12. The elongate member of claim 1 wherein the vapor generator is disposed in a handle on a proximal end of the elongate member.

13. The elongate member of claim 1 wherein the delivery lumen and active insulation lumen are concentric.

14. The elongate member of claim 13 wherein the active insulation lumen is annular.

15. The elongate member of claim 1 wherein an inflow portion of the active insulation lumen, an outflow portion of the active insulation lumen, and the delivery lumen are concentric.

16. The elongate member of claim 1 wherein the active insulation lumen comprises a plurality of inflow and outflow lumens.

17. The elongate member of claim 16 wherein the inflow and outflow lumens are radially outward from the delivery lumen.

18. The elongate member of claim 16 wherein the inflow and outflow lumens are approximately the same distance from the delivery lumen.

19. The elongate member of claim 1 further comprising a needle disposed on a distal end of the delivery lumen.

20. A method of insulating vapor to be delivered to a patient, comprising:
positioning an elongate member within a target tissue in the patient;
delivering vapor through a delivery lumen of the elongate member to the target tissue; and
actively forming a vacuum in an active insulation lumen of the elongate member to insulate the vapor.

21. The method of claim 20 wherein the delivering step further comprises generating the vapor in the delivery lumen.

22. The method of claim 20 wherein the delivering step further comprises generating the vapor outside of the patient.

23. The method of claim 20 wherein the delivering step further comprises generating the vapor in a handle of the elongate member.

24. The method of claim 20 wherein the vacuum is formed with a vacuum device.

25. The method of claim 20 wherein the target tissue is a vein.

26. The method of claim 20 wherein the target tissue is an artery.

27. The method of claim 20 wherein the target tissue is a gland duct.

28. The method of claim 20 wherein the delivering step further comprises delivering vapor through a needle disposed on a distal end of the elongate member.

29. A method of insulating vapor to be delivered to a vein in a patient's body, comprising:
positioning an elongate member within the vein of the patient;
delivering vapor through a delivery lumen of the elongate member to the vein; and
actively insulating the vapor with an active insulation lumen of the elongate member.

30. The method of claim 29 wherein the actively insulating step comprises actively insulating the vapor with a vacuum in the active insulation lumen of the elongate member.

31. The method of claim 29 wherein the vacuum is formed with a vacuum device.

32. The elongate member of claim 31 wherein the vacuum device is a vacuum pump.

33. The elongate member of claim 31 wherein the vacuum device is a wall vacuum.

34. The method of claim 29 wherein the actively insulating step comprises actively insulating the vapor with a gas in the active insulation lumen of the elongate member.

35. The method of claim 34 wherein the gas flows in a distal direction through the active insulation lumen adjacent to the delivery lumen and returns in a proximal direction through the active insulation lumen adjacent to an external surface of the elongate member.

36. The method of claim 29 wherein the actively insulating step comprises actively insulating the vapor with a fluid in the active insulation lumen of the elongate member.

37. The method of claim 29 wherein the delivering step further comprises generating the vapor in the delivery lumen.

38. The method of claim 29 wherein the delivering step further comprises generating the vapor outside of the patient.

39. The method of claim 29 wherein the delivering step further comprises generating the vapor in a handle of the elongate member.

40. The method of claim 29 wherein the delivering step further comprises delivering vapor through a needle disposed on a distal end of the elongate member.
